(19) United States
(12) Patent Application Publication
(10) Pub. No.: US 2006/0251702 A1
(43) Pub. Date: Nov. 9, 2006

(54) Implantable materials and methods for inhibiting tissue adhesion formation

(75) Inventors: Abram D. Janis, Valencia, CA (US); Michael C. Hiles, West Lafayette, IN (US); Jason P. Hodde, West Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Cook Biotech Incorporated, West Lafayette, IN (US)

(21) Appl. No.: 11/429,662

(22) Filed: May 5, 2006

Related U.S. Application Data
(60) Provisional application No. 60/678,533, filed on May 5, 2005.
(60) Provisional application No. 60/678,532, filed on May 6, 2005.

(51) Int. Cl. A61F 2/00; A61K 31/192

(52) U.S. Cl. 424/426; 514/569

(57) ABSTRACT

Described are materials and methods for inhibiting the formation of tissue adhesions. In one aspect, a prosthetic tissue support mesh, and especially such a mesh comprised of a remodelable material that promotes tissue ingrowth, incorporates an effective amount of an anti-inflammatory compound such as a non-steroidal anti-inflammatory drug (NSAID) to inhibit the formation of tissue adhesions to the mesh and/or to surrounding tissues when implanted in a patient. Also described are materials and methods for increasing the length of persistence of implanted resorbable materials, and especially implanted bioremodelable materials, using an anti-inflammatory compound such as an NSAID.

### REFERENCE TO RELATED APPLICATION

This application claims the benefit of priority of U.S. Provisional Patent Application Ser. No. 60/678,533 filed on May 5, 2005, and U.S. Provisional Patent Application Ser. No. 60/678,532 filed May 6, 2005, each of which is hereby incorporated by reference herein in its entirety.

### BACKGROUND

The present invention relates generally to medical devices and procedures. In more particular aspects, the present invention relates to implantable medical materials that provide resistance to the formation of tissue adhesions.

As further background, tissue adhesions can occur during the initial phases of the healing process after surgery or disease. Tissue adhesions are abnormal tissue linkages which can impair bodily function, produce infertility, obstruct the intestines and other portions of the gastrointestinal tract (bowel obstruction) and produce general discomfort. Most commonly, adhesions occur as a result of surgical interventions, although adhesions may also occur as a result of disease, mechanical injury, radiation treatment and the presence of foreign material.

In certain situations, adhesions can pose particular difficulty when using an implantable biomaterial such as such as a prosthetic mesh, e.g. in the repair of hernias or other tissue defects. Prosthetic meshes, such as polypropylene, have historically been used as a support structure for such wound and tissue repair. Unfortunately, however, when using prosthetic mesh, adhesions can form between intraperitoneal structures, such as bowel and omentum, and the repair site. Additionally, the repair site often exhibits irregular or inadequate cellular infiltration and neovascularization, resulting in excessive scarring and a thin tissue layer that is more susceptible to infection or other additional damage. Additionally, wound cavities are often created by raising soft tissue flaps which, after closure, lie directly adjacent to the support material. These wound cavities leak serous fluid and ooze blood which leads to seroma and hematoma formation. As a result, re-operative abdominal surgery is frequently required to repair the complications resulting from the adhesions.

Currently, complications from adhesions are reported to result in 2% of all surgical admissions. Peritoneal adhesions to the ovaries, fallopian tubes, and uterus are responsible for 15-20% of female infertility. As a result of these and other incidences in which adhesions arise, significant economic costs are incurred not only for surgeon and hospital fees, but also in follow-up outpatient care, lost workdays, or the indirect costs of morbidity or mortality.

Various attempts have been made to prevent adhesions. These have included for example the use of peritoneal lavage, heparinized solutions, procoagulants, modification of surgical techniques such as the use of microscopic or laparoscopic surgical techniques, the elimination of talc from surgical gloves, the use of smaller sutures and the use of physical barriers (membranes, gels or solutions) aiming to minimize apposition of serosal surfaces. Specific barrier materials that have been used include, for example, cellulosic barriers, polytetrafluoroethylene materials, and dextran solutions. However, limited success has been experienced with methods used to date.

In view of the background in this area, needs remain for improved or alternative medical materials and methods that may be used to discourage or reduce the formation of adhesions. The present invention is addressed to these needs.

### SUMMARY OF THE INVENTION

Accordingly, in certain aspects, the present invention provides unique medical materials and methods that involve the effective local and targeted delivery of anti-inflammatory compounds such as non-steroidal anti-inflammatory compounds upon a prosthetic mesh material so as to reduce tissue adhesion formation to the prosthetic mesh material. Further, it has been found that such delivery of such anti-inflammatory compounds can even be effectively used to significantly reduce adhesion formation to prosthetic mesh materials that promote tissue infiltration, e.g. in the case of prosthetic mesh materials that comprise a remodelable material such as a remodelable extracellular matrix material.

Accordingly, in one embodiment of the invention, provided is a medical implant material for providing tissue support at an implant site that includes a remodelable extracellular matrix layer that is effective to promote tissue ingrowth into the layer, and an effective amount of an anti-inflammatory compound, and especially a non-steroidal anti-inflammatory compound, to inhibit the formation of adhesions at the implant site.

In further embodiments, the present invention provides methods for supporting patient tissue which include implanting a tissue support material in a patient so as to provide tissue support, wherein the tissue support material includes an effective amount of an anti-inflammatory compound such as a non-steroidal anti-inflammatory drug to inhibit the formation of tissue adhesions. In some forms of the invention, the tissue support is provided in the repair of a hernia such as an inguinal hernia, and the non-steroidal anti-inflammatory compound effectively inhibits the development of abdominal adhesions. In such methods, the tissue support material can have the drug immobilized on only one side, and that side can be secured facing the adhesiogenic tissue, such as bowel tissue. In other forms of the invention, the tissue support material is deployed between tissue planes, for instance as a suture cover for an abdominal surgical incision or otherwise, and can inhibit the formation of adhesions between the tissue planes. For such deployments, advantageous forms of the tissue support material will have amounts of the drug immobilized on both sides of the material, for example either as surface coatings or homogenously distributed through the material.

In another aspect, the present invention provides a method of manufacturing an adhesion-inhibited medical tissue support mesh material. This method includes providing a tissue support mesh material, and incorporating on the material an effective amount of an anti-inflammatory compound, and especially a non-steroidal anti-inflammatory drug, to inhibit the formation of tissue adhesions.

Still a further embodiment of the invention provides a barrier material for interposition between adhesiogenic tissue and another structure such as a tissue or implant material, to inhibit adhesion formation. The barrier material of the invention includes an implantable, desirably biodegradable barrier sheet, and an effective amount of an anti-inflammatory compound such as a non-steroidal anti-inflammatory drug compound to inhibit the formation of adhesions. The non-steroidal anti-inflammatory drug or other compound can be carried by the sheet in any suitable fashion including for example incorporation homogeneously throughout the material forming the sheet, and/or incorporated directly on one or both faces of the sheet, or in a carrier layer applied to sheet.

In another embodiment, the present invention provides a method for inhibiting tissue adhesions in a patient which includes interposing a barrier sheet material between an adhesiogenic tissue and another structure, such as an implant and/or other tissue, wherein the barrier sheet material includes an effective amount of an anti-inflammatory agent and especially a non-steroidal anti-inflammatory drug compound to increase resistance to tissue adhesions between the adhesiogenic tissue and the other structure.

In additional embodiments of the invention, NSAID or other anti-inflammatory compounds are used to delay the resorption, or increase the persistence over time, of implanted resorbable materials, and in preferred embodiments, implanted bioremodelable materials. This can be used, for example, in tissue support applications wherein the material is implanted to support soft tissues, and an enhanced retention of material strength is desired. Illustratively, in certain embodiments, an interior region (e.g. interior layers of a multilaminate construct) can be loaded with a sufficient level of NSAID to delay resorption, while an exterior region lacks the NSAID or has relatively lower amounts. In this fashion, desired tissue integration into outer layers or regions of the implanted material can be facilitated, while inner layers or regions persist to provide strength. Such embodiments are advantageously carried out with remodelable implant materials, and especially remodelable ECM materials.

These and other embodiments as well as features and advantages of the present invention will be apparent from the descriptions herein.

### DESCRIPTION OF THE FIGURES

FIG. 1 provides an illustration of a tissue support device of the invention in use to repair a hernia.

FIG. 2 provides a sectional view of an illustrative multilaminate device of the present invention.

FIG. 3 provides an illustration of a barrier layer device of the invention interposed between adhesiogenic bowel tissue and abdominal wall tissue.

FIG. 4 provides an illustration of an illustrative tubular implant device of the present invention grafted to a native tubular vessel.

FIGS. 5-16 provide charts setting forth data generated in the Examples below.

### DETAILED DESCRIPTION

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

As disclosed above, the present invention provides medical materials that include a remodelable extracellular matrix layer in combination with an effective, tissue adhesion-inhibiting amount of a non-steroidal anti-inflammatory drug (NSAID), as well as methods of manufacturing and using such materials. The present invention also provides adhesion-inhibited tissue support meshes that incorporate immobilized amounts of an NSAID compound, and methods for their manufacture and use. In addition, in other aspects, the present invention provides barrier materials that incorporate an effective amount of an NSAID compound, wherein the barrier materials can be interposed between adhesiogenic tissues and other structures such as implants or other tissue, so as to inhibit the formation of tissue-adhesions. Related barrier methods and manufacturing processes represent additional embodiments of the present invention.

Turning now to a discussion of non-steroidal anti-inflammatory drugs that may be used in the invention, a wide variety of such drugs are known and will be suitable. Many of these drugs modulate prostaglandin synthesis by inhibiting cyclooxygenases that catalyze the transformation of arachidonic acid, which is the first step in the prostaglandin synthesis pathway. It is currently understood that two cyclooxygenases are involved in the transformation of arachidonic acid, and these have been termed cyclooxygenase-1 (COX-1) and cyclooxygenase-2 (COX-2). COX-1 is a constitutively produced enzyme involved in many of the non-inflammatory regulatory functions related to prostaglandins. COX-2, on the other hand, is an inducible enzyme with significant involvement in the inflammatory process. Inflammation causes the induction of COX-2, leading to the release of prostanoids, which sensitize peripheral nociceptor terminals and produce localized pain hypersensitivity.

Nonsteroidal anti-inflammatory drugs (NSAIDs) constitute a wide range of pharmacologically active agents with diverse chemical structures. Some chemical classes of NSAIDs include: a) salicyclic acid derivatives (e.g., aspirin), b) phenylacetic acids (e.g., diclofenac), c) heterocyclic acetic acids (e.g., indomethacin, sulindac), d) proprionic acids (e.g., ibuprofen, naproxen), e) fenamic acids (e.g., flufenamic), f) pyrazolones (e.g., phenylbutazone), and g) oxicams (e.g., piroxicam). Among all NSAIDs, the common mechanism of action for their anti-inflammatory properties resides in their ability to inhibit cyclooxygenase, an enzyme required for prostaglandin synthesis.

In certain inventive embodiments, the NSAID compound utilized in the present invention is a non-selective COX inhibitor that substantially inhibits both COX-1 and COX-2. In other embodiments, the NSAID compound used is a selective COX-1 inhibitor. In still further inventive aspects, the NSAID compound is a selective COX-2 inhibitor.

There are a variety of methods that can be used to evaluate the COX selectivity of a compound. For example, a number of in vitro and ex vivo (e.g., whole blood) assays have been established to determine the IC₅₀ (μM) value of various compounds (see Brooks et al. (1999) Br. J. Rheumatol. 38, 779-88, Warner et al. (1999) Proc. Natl. Acad. Sci. USA 96, 7563-8, Brideau et al. (1996) Inflamm. Res. 45, 68-74 and Patrignani et al. (1994) J. Pharmacol. Exp. Ther. 271, 1705-1712 and Pairet (1998) J. Clin. Rheum. 4, S17-25). The IC₅₀ value represents the concentration at which the compound achieves 50% of its maximal inhibition of COX. It is well known that NSAIDS vary in their ability to inhibit both isoforms of COX. Consequently, it has become routine practice to define a compound's selectivity for either isoform of COX as a ratio of their respective IC₅₀ values (IC_{50COX-2}/IC_{50COX-1}) Compounds with values significantly less than 1 are said to have selectivity for COX-2. Conversely, compounds with ratios significantly greater than 1 are said to be COX-1 selective, while those with ratios essentially equal to 1 are non-selective. Although IC₅₀ values of COX-1 and COX-2 said to be in both humans and animals have been reported for a variety of compounds, it is well understood in the art that ratios of the same compound may vary somewhat depending on the selectivity assay used to generate these IC₅₀ values. However, those skilled in the art have developed generally-accepted classifications, and given the teachings herein will recognize the ability to use COX-2 selective, COX-1 selective, and non-selective COX inhibitors in aspects of the present invention.

A variety of nonselective NSAID COX inhibitors are known, and include, for example, aspirin, ibuprofen, indomethacin, ketorolac, naprosen, oxaprosin, tenoxicam and tolmetin. Many COX-1 selective NSAIDs are also known, and include but are not limited to flurbiprofen, ketoprofen, fenoprofen, piroxicam and sulindac.

More recently, compounds that selectively inhibit COX-2 as compared to COX-1 have been discovered. Numerous relatively COX-2 selective inhibitors are known, and include but are not limited to diclofenac, etodolac, meloxicam, nabumetone, nimesulide (N—C4-nitro-2-phenoxyphenyl methanesulfonamide and 6-MNA. A variety of highly selective COX-2 inhibitors are also known, and include celecoxib, rolfecoxib and other drugs such as L-743337, NS-398 and SC 58125.

Additional information concerning these and other selective COX-2 NSAID compounds can be found in the patent and other literature on the subject, including for instance: celecoxib (CAS RN 169590 51 C-27791 SC-586531 and in U.S. Pat. No. 5,466,823); deracoxib (CAS RN 169590 4); rofecoxib (CAS RN. 162011 7); compound B-24 (U.S. Pat. No. 5,840,924); compound B-26 (WO 00/25779); and etoricoxib (CAS RN 202409 4, MK-663, SC-86218, and in TahIP 2); parecoxib (see U.S. Pat. No. 5,932,598) is a therapeutically effective prodrug of the tricyclic COX-2 inhibitor valdecoxib, (U.S. Pat. No. 5,633,272) and can be employed as a source of a cyclooxygenase inhibitor, including as its sodium salt; compound ABT-963 described in International Publication number WO 00/24719 is a tricyclic COX-2 inhibitor; phenylacetic acid derivative COX-2 inhibitors can be used, including those described in WO 99/11605 such as the compound designated as COM 89 (CAS RN 346670 4); compounds that have similar structures are described in U.S. Pat. Nos. 6,310,099 and 6,291,523; information as to N-(2-cyclohexyloxynitrophenyl)methane sulfonamide (NS-398, GAS RN 123653 2) can be found in Yoshimi, N. et al., Japanese J. Cancer Res., 90(4).R406-412 (1999), in Falgueyret, J.-P et al., Science Spectra, available at: hftp://www.gbhap.com/Science,Spectra/20article.htm (Jun. 6, 2001), and in Iwata, K. et al., Jpn. J. Pharmacol., 75(2):191-194 (1997); other COX-2 inhibitors include diarylmethylidenefuran derivatives described in U.S. Pat. No. 6,180,651.

Additional known selective COX-2 inhibitors include N-(2-cyclohexyloxynitrophenyl)methane sulfonamide; (E)[(4methylphenyl)(tetrahydro oxo furanylidene)methyl]benzenesulfonamide; darbufelone (Pfizer); CS-502 (Sankyo); LAS 34475 (Almirall Profesfarna); LAS 34555 (Almirall Profesfarma); S-33516 (Servier, see Current Drugs Headline News, at hftp://www.current-drugs, com/NEWS/Inflal.htm, Oct. 4, 2001); 1 BMS-347070 (Bristol Myers Squibb, described in U.S. Pat. No. 6,180,651); MK-966 (Merck); L783003 (Merck); T-614 (Toyama); D-1 367 (Chiroscience); L-748731 (Merck); CT3 (Atlantic Pharmaceutical); CGP-28238 (Novartis); BF-389 (Biofor/Scherer); CR253035 (Glaxo Wellcome); 6-dioxo-9H-purin yi-cinnamic acid (Glaxo Wellcome); S-2474 (Shionogi); compounds described in U.S. Pat. Nos. 6,310,079; 6,306,890 and 6,303,628 (bicycliccarbonyl indoles), U.S. Pat. No. 6,300,363 (indole compounds), U.S. Pat. Nos. 6,297,282 and 6,004,948 (substituted derivatives of benzosulphonamides), U.S. Pat. Nos. 6,239,173, 6,169,188, 6,133,292, 6,020,343, 6,071,954, and 5,981,576 ((methylsulfonyl)phenyl furanones), U.S. Pat. No. 6,083,969 (diarylcycloalkano and cycloalkeno pyrazoles), U.S. Pat. No. 6,222,048 (diaryl(51-1)-furanones), U.S. Pat. No. 6,077,869 (aryl phenylhydrazines), U.S. Pat. Nos. 6,071,936 and 6,001,843 (substituted pyridines), U.S. Pat. No. 6,307,047 (pyridazinone compounds), U.S. Pat. No. 6,140,515 (3-aryl aryloxyfuranones), U.S. Pat. Nos. 6,204,387 and 6,127,545 (diaryl pyridines), U.S. Pat. No. 6,057,319 (314diar@hydroxy-2,5-dihydrofurans), U.S. Pat. No. 6,046,236 (carbocyclic sulfonamides), U.S. Pat. Nos. 6,002,014, 5,994,381 and 5,945,539 (oxazole derivatives), and U.S. Pat. Nos. 6,034,256 and 6,077,850 (Benzopyran derivatives).

Preferred COX-2 inhibitors for use in the present invention include nimesulide, celecoxib (Celebrex™), rofecoxib (Vioxx™), meloxicam, piroxicam, deracoxib, parecoxib, valdecoxib (Bextra™), etoricoxib, a chromene derivative, a chroman derivative, N-(2cyclohexyloxynitrophenyl)methane sulfonamide, COX1 89, ABT963, JTE-522, pharmaceutically acceptable salts, prodrugs or mixtures thereof.

Nimesulide is an especially preferred NSAID for use in aspects of the present invention, although as taught herein NSAIDs other than nimesulide can also be used.

In certain aspects of the invention, a non-acidic NSAID (i.e. and NSAID compound having a pKa of 7 or above when dissolved in water) will be used in accordance with the teachings herein to inhibit tissue adhesions. Suitable non-acidic NSAID compounds for these purposes include, by way of example, nimesulide, celecoxib, and rofecoxib. In addition, or alternatively, the NSAID compound used in the present invention can be insoluble in water or have a level of water solubility that is otherwise sufficiently low that substantially no dissolution of the compound in the biological fluids at the implant site occurs which would cause undesired levels of migration of amounts of the NSAID compound from the implanted material and/or site as dissolved molecular species. Suitable substantially water-insoluble NSAID drug materials which can be used in accordance with this aspect of the invention include, but are not necessarily limited to, nimesulide, celecoxib, rofecoxib, naproxen, ibuprofen, sulindac, diclofenac, fenclofenac, alclofenac, ibufenac, isoxepac, furofenac, tiopinac, zidometacin, acemetacin, fentiazac, clidanac, oxipinac, zomepirac sodium and pharmaceutically acceptable water-insoluble salts thereof.

As noted above, in certain embodiments of the invention, the NSAID compound will be applied to a bioremodelable medical material in an amount effective to decrease adhesions when the material is implanted in a patient. In this regard, a bioremodelable material as identified herein will be one which possesses the capacity to promote tissue ingrowth into the material as it is resorbed.

Bioremodelable materials are used to advantage in certain medical devices and methods of the present invention, particularly bioremodelable collagenous materials. Such bioremodelable collagenous materials can be provided, for example, by collagenous materials isolated from a suitable tissue source from a warm-blooded vertebrate, and especially a mammal. Such isolated collagenous material can be processed so as to have bioremodelable properties and promote cellular invasion and ingrowth. Bioremodelable materials may be used in this context to promote cellular growth within the site in which a medical device of the invention is implanted.

Suitable bioremodelable materials can be provided by collagenous extracellular matrix materials (ECMs) possessing biotropic properties. Illustrative suitable extracellular matrix materials for use in the invention include, for instance, submucosa (including for example small intestinal submucosa, stomach submucosa, urinary bladder submucosa, or uterine submucosa, each of these isolated from juvenile or adult animals), renal capsule membrane, dermal collagen, amnion, dura mater, pericardium, serosa, peritoneum or basement membrane materials, including liver basement membrane or epithelial basement membrane materials. These materials may be isolated and used as intact natural forms (e.g. as sheets), or reconstituted collagen layers including collagen derived from these materials and/or other collagenous materials may be used. For additional information as to submucosa materials useful in the present invention, and their isolation and treatment, reference can be made to U.S. Pat. Nos. 4,902,508, 5,554,389, 5,733,337, 5,993,844, 6,206,931, 6,099,567, and 6,331,319. Renal capsule membrane can also be obtained from warm-blooded vertebrates, as described more particularly in International Patent Application serial No. PCT/US02/20499 filed Jun. 28, 2002, published Jan. 9, 2003 as WO03002165.

In some embodiments of the invention, an isolated ECM or other collagenous material for use in the invention is prepared in such a manner that it retains growth factors and/or other bioactive components native to the source tissue. For example, submucosa or other ECMs may include one or more growth factors such as basic fibroblast growth factor (FGF-2), transforming growth factor beta (TGF-beta), epidermal growth factor (EGF), platelet derived growth factor (PDGF), vascular endothelial growth factor (VEGF), and/or connective tissue growth factor (CTGF). As well, submucosa or other ECM when used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression. In particular aspects, the ECM material will exhibit the capacity to induce angiogenesis when implanted in a human or other mammalian patient.

Further, in addition or as an alternative to the inclusion of such native bioactive components, non-native bioactive components such as those synthetically produced by recombinant technology or other methods, may be incorporated into the material used for the covering. These non-native bioactive components may be naturally-derived or recombinantly produced proteins that correspond to those natively occurring in an ECM tissue, but perhaps of a different species (e.g. human proteins applied to collagenous ECMs from other animals, such as pigs). The non-native bioactive components may also be drug substances.

Submucosa or other ECM tissue used in the invention can be highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

Isolated ECM or other biocompatible layers can be used in the invention as single layer constructs, but in certain advantageous embodiments will be used in multilaminate constructs. In this regard, a variety of techniques for laminating layers together are known and can be used to prepare multilaminate constructs used in the present invention. For example, a plurality of (i.e. two or more) layers of collagenous material, for example submucosa-containing or other ECM material, can be bonded together to form a multilaminate structure. Illustratively, two, three, four, five, six, seven, or eight or more collagenous layers containing submucosal or other collagenous ECM materials can be bonded together to provide a multilaminate collagenous substrate material for use in the present invention. In certain embodiments, two to eight collagenous, submucosa-containing layers isolated from intestinal tissue of a warm-blooded vertebrate, particularly small intestinal tissue, are bonded together. Porcine-derived small intestinal tissue is preferred for this purpose. The layers of collagenous tissue can be bonded together in any suitable fashion, including dehydrothermal bonding under heated, non-heated or lyophilization conditions, using adhesives, glues or other bonding agents, crosslinking with chemical agents or radiation (including UV radiation), or any combination of these with each other or other suitable methods. For additional information as to multilaminate ECM constructs that can be used in the invention, and methods for their preparation, reference may be made for example to U.S. Pat. Nos. 5,711,969, 5,755,791, 5,855,619, 5,955,110, 5,968,096, and to U.S. Patent Publication No. 20050049638 A1 published Mar. 3, 2005. These constructs can be perforated or non-perforated, and when perforated may include an array of perforations extending substantially across the surface of the construct, or may include perforations only in selected areas such as adjacent to the periphery of the construct and configured to provide suture holes for attachment of the construct to tissue to be supported.

The non-steroidal anti-inflammatory drug can be carried by the ECM or other sheet material in any suitable fashion. For example, the drug can be a powder which is applied, by spraying, rubbing or otherwise, to one or both sides of the sheet. The drug may also be applied in the form of a liquid medium containing the drug, such as a solution or suspension, which is contacted with all or only one or more portions of the sheet, after which the sheet can be dried to leave the drug. Contact between the liquid medium and the sheet can be achieved in any suitable manner, including for example immersion, spraying or otherwise. After drying, the drug may be substantially homogenously dispersed through the sheet, or may be selectively applied to regions of the sheet.

In certain embodiments of the invention, the anti-inflammatory drug is applied selectively to one side of a generally planar sheet, while the other side lacks the drug or has a lesser amount of the drug. In this fashion, directional post-surgical adhesion resistance can be provided, for instance in situations wherein tissue invasion or attachment to an opposite side of the sheet is desired, and/or wherein damaged tissue resides adjacent to the opposite side of the sheet when implanted, and delivery of the anti-inflammatory compound into the damaged tissue, which may impair healing processes, is to be minimized or prevented. Such situations can include the repair of hernias of the abdominal wall with a tissue support mesh of the invention, wherein the NSAID-loaded side of the sheet is positioned facing the interior of the abdomen (e.g. toward bowel tissue within the abdomen). In this manner, preferential release of the NSAID toward the relatively adhesiogenic tissue can be provided, while minimizing or avoiding release in the opposite direction where adhesion formation presents a lesser or no significant risk.

Further in this regard, biocompatible sheet material used in the invention may be permeable or impermeable to water. Where water-impermeable materials are used, amounts of the NSAID compound selectively coated on one side of the material can be effectively prevented from migration through the material to and out the other side. Where water-permeable biocompatible sheet materials are used, some amount of the NSAID compound selectively coated on one side of the material can migrate through the material and out the opposite side. In variants of the present invention, a water-permeable biocompatible sheet material will have a coating or bonded layer on one side thereof that is water-impermeable or less water-permeable than the biocompatible sheet material, wherein the sheet material has the NSAID compound applied selectively to the other side thereof and/or distributed within the sheet material. In this manner, improved preferential release of the NSAID compound to one side of the biocompatible sheet material can be achieved.

Still further, some biocompatible sheet materials show greater permeability to water in a direction from a first side to a second side, than in the opposite direction. In such differentially permeable materials, the NSAID can be selectively coated on the second side to provide a reduced level or likelihood of NSAID migration through the sheet as compared to coating the sheet on the first side. Illustratively, many ECM materials demonstrate such differential permeability. As one example, small intestine submucosa, unless its native porous structure is altered or blocked, exhibits greater permeability to water in a direction from its abluminal side (first side) to its luminal side (second side). In certain inventive constructs, the NSAID compound is thus selectively coated on the luminal side of a layer of small intestine submucosa.

In some embodiments of the invention, the biocompatible sheet device is configured to release the NSAID compound from both sides. For example, the NSAID on such devices can be coated on both sides and/or distributed homogeneously or otherwise through the thickness of the sheet material. Such devices may be used to advantage in surgical procedures in which the device is to be implanted into and/or between tissue planes. Illustratively, such devices can serve beneficially as suture covers for abdominal or other surgical incisions, and can inhibit the formation of adhesions between tissue planes.

As noted above, certain embodiments of the invention provide tissue supporting meshes or sheets that incorporate an effective tissue adhesion-preventing amount of a non-steroidal anti-inflammatory drug. Generally, tissue-supporting mesh devices of the invention will have sufficient strength to provide beneficial support to tissues to which they are attached. Tissue support mesh devices of the invention can have sufficient strength to effectively retain sutures or other surgical fasteners, for example exhibiting a suture retention strength of at least about 100 gram force, e.g. in the range of about 100 to about 1000 gram force, and more typically in the range of about 200 to about 600 gram force, each of these based upon 5-0 Prolene suture and a bite depth of 2 mm, and a hydrated material condition in the case of ECM or other wettable materials. Suitable tissue support meshes for use in these embodiments of the invention can be made from single layer or multilaminate ECM materials as discussed above.

In other inventive variants, the tissue support mesh is comprised of a non-biodegradable synthetic polymeric material. Suitable such synthetic polymeric materials for these purposes include for example polyproplylene, polytetrafluoroethylene (PTFE), polyamide (e.g. Nylon), polyester, or other suitable biocompatible polymers. Such tissue support mesh devices of the invention may advantageously include knitted polypropylene monofilament mesh fabrics such as those available from Ethicon, Inc. under the Prolene trademark, as well as meshes available from Ethicon, Inc. under the Vicryl trademark. Other tissue support mesh materials useful in the invention include those available under the Marlex, Dacron, Teflon and Merselene trademarks.

The NSAID compound can be applied directly onto and/or within the support mesh as discussed above, or can be incorporated in a carrier layer adhered to the mesh. In variants of the invention, the support mesh is formed with a synthetic polymer, and a carrier layer containing the anti-inflammatory drug is applied to one or both sides of the support mesh. The carrier layer can effectively release the drug to the surrounding environment, can retain the drug within the layer to affect invading tissue, or combinations thereof.

When present, the carrier layer may be formed of any suitable material. In certain embodiments, the carrier layer is formed with a non-biodegradable material; in others, it is advantageously formed with a biodegradable material. Illustrative biodegradable carrier layer-forming materials include synthetic polymers and/or naturally-occurring polymers, including by way of example polylactic acid homopolymers, polyglycolic acid homopolylmers, copolymers of polylactic acid and polyglycolic acid, polycaprolactone, polyanhydrides, polypeptide materials such as gelatin or collagen, and hydroxymethyl cellulose, to name a few. These materials can be obtained commercially or can be prepared using techniques known to the art.

The NSAID compound can be incorporated into the carrier layer in any suitable fashion. In certain embodiments, the NSAID compound is incorporated substantially homogenously into the carrier layer, for example by distributing the compound in a flowable or workable mixture which is then caused to form the carrier layer. Such workable mixtures may include for instance a polymerizable monomer preparation which is then polymerized to form the carrier layer, a molten polymer preparation which is then cooled to form the carrier layer, a non-crosslinked flowable polymer preparation which is then crosslinked to form the carrier layer, or a solvated or dispersed film-forming polymer preparation which is then dried to form the carrier layer. The practice of these and other modes for forming the carrier layer incorporating the NSAID compound will be within the purview of those skilled in the art given the teachings herein.

As well, the carrier layer may be formed directly upon a tissue support mesh substrate to adhere to the same, or may be formed separately and then attached to the sheet substrate, illustratively using suitable bonding agents or techniques. In this regard, such independently-formed layers incorporating the NSAID compound in an effective tissue-adhesion inhibiting amount also form a part of the present invention. Such layers can be used as layers interposed between adhesion-forming tissues and other structures to be protected, and in so doing can serve both as physical barriers to adhesion formation and to deliver active, adhesion-inhibiting NSAID compounds. The NSAID-containing layers, whether formed upon another substrate sheet or formed and/or used independently, may have a thickness ranging from about 100 micrometers to about 1 millimeter, more typically from about 300 micrometers to about 500 micrometers.

The NSAID compound will be included in the sheet or layer of the invention in an amount which is effective to decrease the extent of and/or the tenacity of tissue adhesions to the sheet or layer itself or to another structure protected by the sheet or layer, e.g. an adjacent tissue structure or implant surface. In certain aspects of the invention, the sheet or layer will include the NSAID compound at a level of about 1 to 100 micrograms per square centimeter (μg/cm²), more typically about 2 to about 40 μg/cm². As to the total dose of NSAID compound delivered, this will depend upon many factors including for instance the particular NSAID employed, the size of the area requiring protection and thus the size of the sheet or layer to be implanted, and other like factors.

In aspects of the invention wherein the NSAID compound is carried by an implant, including a bioremodelable implant such as a bioremodelable ECM, the implant may have a generally planar form or may have a more three-dimensional form such as a tube. Tube-shaped implants, for example vascular graft implants, can have the NSAID carried upon inner and/or outer surfaces. In particular aspects of the invention, such tubular implants will have only their exterior surfaces coated with the NSAID compound or a carrier layer containing the NSAID compound, to decrease the extent and/or tenacity of tissue adhesions to the exterior surfaces of the implant.

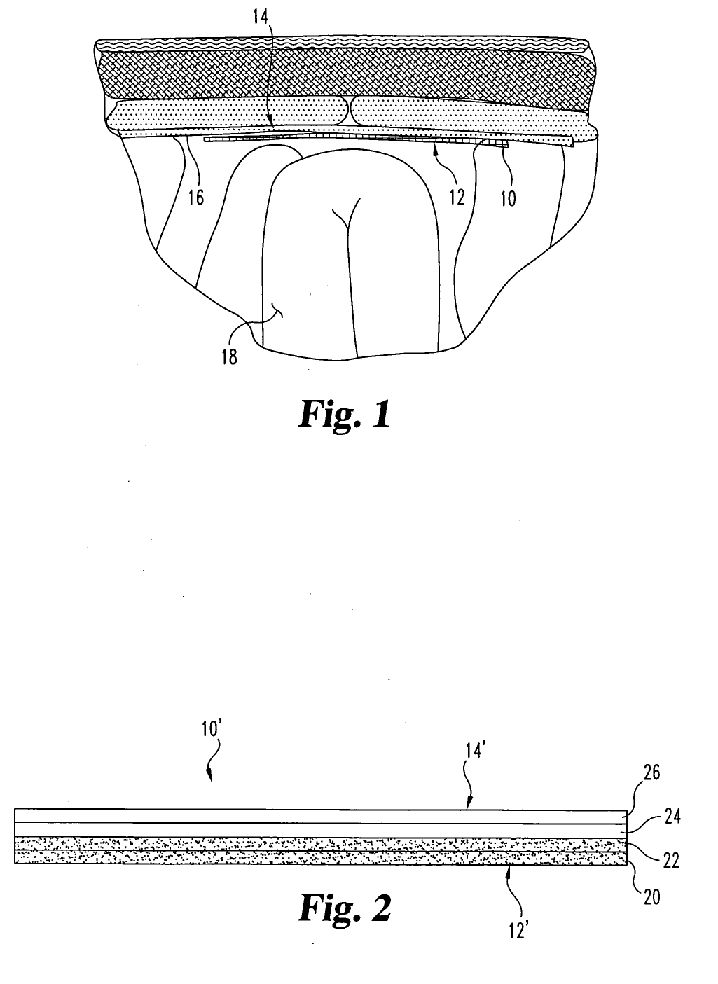

With reference now to the Figures, shown in FIG. 1 is an illustrative tissue support mesh implant 10 positioned within a patient to treat a hernia. Implant 10 is a generally planar tissue support mesh device having a first side 12 and a second side 14. Implant 10 is secured to the inside of abdominal wall 16 in the repair of the hernia. In advantageous embodiments of the invention, the first side 12 of the implant 10 is coated with or has a carrier layer containing an adhesion-inhibiting amount of an NSAID compound, whereas second side 14 has no such coating or carrier layer. In this fashion, the NSAID compound will be preferentially retained and/or released toward the bowel tissue 18 and will inhibit the formation of bowel tissue adhesions. The second side 14 of the implant 10 will contain or release little or no NSAID compound, and thus will tend not to cause interference with wound healing in the herniated and surgically repaired tissue that would be caused by the presence of substantial levels of the NSAID compound. In preferred aspects, implant 10 is a remodelable tissue support mesh, such as a remodelable ECM sheet device. In this manner, while the NSAID compound effectively inhibits adhesion formation on one side of the implant 10, the implant 10 can contact and promote healing of and/or form adhesions with tissue on the other side, such as mesentery or body wall tissues.

In this regard, with reference to FIG. 2, such an ECM sheet device 10′ can be a multilaminate ECM device including for example from two to about ten isolated ECM layers. Particularly preferred ECM layer materials for these purposes are submucosa-containing ECM layer materials such as those described above, including particularly small intestine submucosa. The ECM sheet device 10′ thus has a first side 12′ and a second side 14′ that are formed by different layers of the multilaminate device. First side 12′ effectively carries effective amounts of the NSAID compound, whereas second side 14′ does not. This can be accomplished using any suitable coating, impregnating or carrier-layer method as discussed above. In certain inventive constructs, this preferential loading of the implant 10′ with the NSAID compound is achieved by selectively impregnating one or more layers at or near side 12′ with the NSAID compound, for example bottom-most two layers 20 and 22 in FIG. 2 (see shading which designates the presence of the NSAID compound). This may be accomplished after formation of the multilaminate construct. In inventive modes, however, this is achieved at least in part, and potentially completely, by impregnating (e.g. by dry powder coating, soaking or spraying) one or more of the ECM layers to be incorporated into the multilaminate construct with the NSAID, and then incorporating those one or more impregnated layers into the construct. In one particular aspect, the one or more NSAID-containing layers are prepared by soaking with a solution or other liquid medium containing the NSAID, and are then layered together with one or more wetted non-NSAID-impregnated layers. The thus-assembled layers can then be dried and bonded together, desirably by dehydrothermal bonding techniques such as vacuum pressing or lyophilization. The resulting construct will thereby be selectively loaded with the NSAID compound toward side 12′.

In other embodiments, multilaminate devices such as that shown as 10′ in FIG. 2 can have NSAID or other anti-inflammatory compound loaded interior layers, and non-loaded exterior layers. The NSAID can then be beneficially retained in the implant and/or can be diffused controllably from the implant from the inner layer(s) and through the outer layer(s). For instance, the central two layers of the device 10′ can be loaded with NSAID, while the outer two layers are not.

Further, a multilaminate ECM device 10′ can be processed such that at least one and potentially all of its layers have a collapsed matrix structure exhibiting reduced porosity and water permeability, thus minimizing or avoiding migration of the NSAID compound through the construct. Accordingly, certain embodiments, all layers 20, 22, 24 and 26 are dried under compression, for instance by vacuum pressing and drying the entire construct, while in other embodiments, one or more but not all of its layers are dried under compression. In one specific embodiment, at least the outermost layer of the NSAID-free side of the construct (layer 26 providing side 14′ in the construct of FIG. 2) can be processed differently and exhibit a higher porosity. For instance, layer 26 and potentially also adjacent layer 24 can be lyophilized or air dried ECM layers, while the remainder of the layers can be compressed/dried (e.g. vacuum pressed) ECM layers.

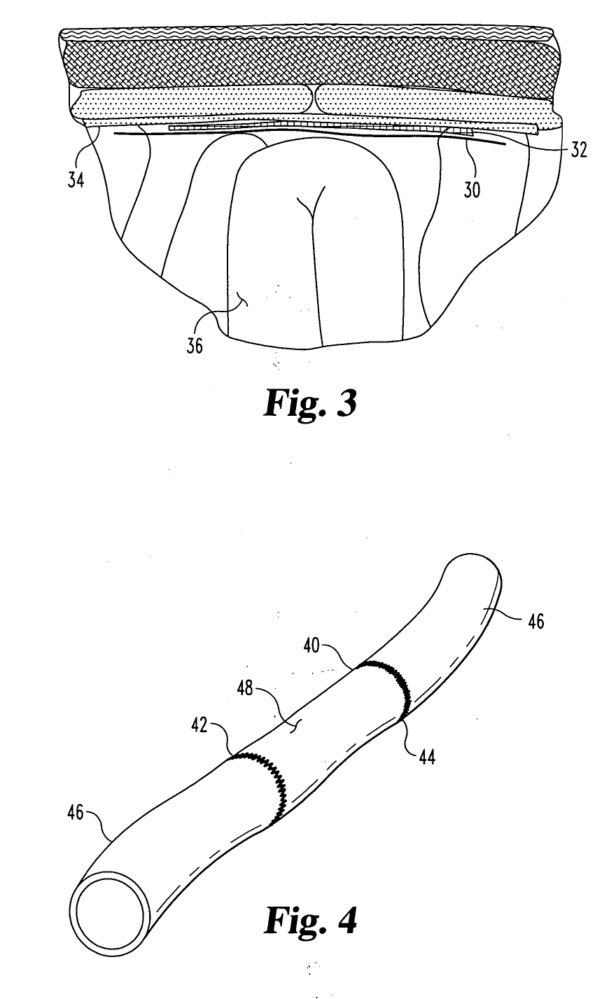

Referring now to FIG. 3, shown is an NSAID-releasing physical barrier sheet device 30 in use to inhibit tissue adhesions after a hernia repair procedure. Sheet device 30 is made of a biodegradable material incorporating a tissue-adhesion inhibiting level of an NSAID compound. Sheet device 30 is not deployed to support tissue and thus does not necessarily possess the strength typical of tissue support meshes, although it can. Device 30 is shown interposed between a hernia mesh 32 attached to the interior surface of the abdominal wall 34 and bowel tissue 36. In this manner, device 30 provides both a physical barrier and localized NSAID compound activity to resist the formation of tissue adhesions between the bowel tissue and abdominal wall and/or mesentary tissue. Device 30 can also release NSAID compound into the bowel tissue region to inhibit the formation of adhesions between bowel segments.

Referring now to FIG. 4, shown is a tubular graft device 40 in accordance with the present invention. Tubular graft device 40 is shown in place having first and second ends 42 and 44 attached to a grafted tubular body structure 46, such as a vascular vessel, e.g. a vein or artery, or another bodily vessel such as the urethra, ureter, or esophagus. Graft device 40 include an outer surface 48 that is coated with or has applied thereto a carrier layer containing an effective amount of the NSAID compound to inhibit tissue adhesions. In this fashion, the tubular bodily structure can be repaired while avoiding or minimizing the formation of adhesions between surrounding tissues and the implanted graft 40.

In other aspects of the invention, it was observed in the testing described below that loading with anti-inflammatory compounds enhanced the persistence of implanted bioremodelable ECM materials. Thus, in additional inventive embodiments, NSAID or other anti-inflammatory compounds are used to delay the bioresorption, or increase the persistence over time, of implanted resorbable materials, and in preferred embodiments, implanted bioremodelable materials. This aspect of the invention can be used, for example, in tissue support applications wherein the material is implanted to support soft tissues, and an enhanced retention of material strength is desired. Illustratively, an interior region (e.g. interior layers of a multilaminate ECM construct as described hereinabove) can be loaded with a sufficient level of NSAID to delay resorption, while an exterior region lacks the NSAID or has relatively lower amounts. In this fashion, desired tissue integration into outer layers or regions of the implanted material can be facilitated, while inner layers or regions persist to provide strength. In this aspect of the invention, the material can be implanted near adhesiogenic tissue as described for other inventive aspects herein, or in regions where adhesiogenesis does not present a significant concern. As well, the NSAID or other anti-inflammatory compound can be included in amounts selected to control the rate of remodeling and thus persistence of the remodelable material, which amounts, in certain instances, can be higher or lower than those needed to achieve significant reductions in tissue adhesion formation as disclosed herein.

The anti-inflammatory compound can be incorporated in the bioremodelable ECM material to reduce the rate of remodeling by incorporating the compound in the material as implanted, and/or the compound (e.g. in solution, suspension or solid form) can be delivered to the implant site upon and/or after the material is implanted. Illustratively, separate NSAID-delivering layers as discussed hereinabove can be implanted in the region to locally deliver anti-inflammatory activity to control the rate of remodeling and increase material persistence. Known local depot forms or other delivery methods, including for instance injection, may also be used.

For the purpose of promoting a further understanding of aspects of the present invention, the following specific examples are provided. It will be understood that these examples are not limiting of the present invention.

### EXAMPLE 1

### Animal Model Testing

This Example describes an animal model used to test the effects of NSAID addition to materials on the formation of tissue adhesions.

Methods

Twenty-nine 250-300 gram Sprague-Dawley rats were anesthetized with an intramuscular (IM) dose of ketamine hydrochloride (90 mg/kg) and xylazine (10 mg/kg) and prepared for aseptic abdominal surgery. Using established techniques (see T. Guvenal et al., 2002 Human Repro 16(8): 1732-51), the cecum was exteriorized, abraded with a nylon brush, and placed back into the abdominal cavity. Additionally, the peritoneal cavity was mildly scraped with a #15 scalpel blade in order to increase the incidence and/or severity of adhesions.

Groups of rats received one of the following 4 treatments:
Treatment 1: (Sham) The cecum was exteriorized, but not abraded. No biomaterials were implanted. N=3 rats.
Treatment 2: (Control) No biomaterial implanted. N=8 rats.
Treatment 3: a lyophilized 2-layer piece of porcine small intestinal submucosa (SIS) was placed over the abraded surface and secured with non-absorbable nylon suture. N=8 rats.
Treatment 4: a piece of polypropylene mesh (Bard) was placed over the abraded surface and secured with non-absorbable nylon suture. N=8 rats.

Following placement of the cecum back into the abdominal cavity, the abdominal wall was closed with 4-0 silk, and the skin closed with surgical staples. Twenty-one days after surgery, the rats were euthanized and the extent and tenacity of adhesions were evaluated using a previously described scale (see M. Oncel et al., 2001 J Surg Res 101(1): 52-53) as follows:

For scoring the extent of adhesions:

0=no adhesion

1=adhesions on 25% of the traumatized cecal surface

2=adhesions on 50% of the traumatized cecal surface

3=adhesions on 75% of the traumatized cecal surface

4=adhesions on 100% of the traumatized cecal surface.

For scoring the tenacity of adhesions:

0=no resistance to separation

1=mild resistance

2=moderate resistance to separation

3=marked resistance

4=sharp dissection required for separation

Statistical Analysis

A nonparametric one-way ANOVA (Kruskal-Wallis) was performed on the extent and tenacity of adhesions to test for overall differences among the treatment groups. This was followed by Wilcoxon tests to compare pairs of individual treatments for significant differences.

Results

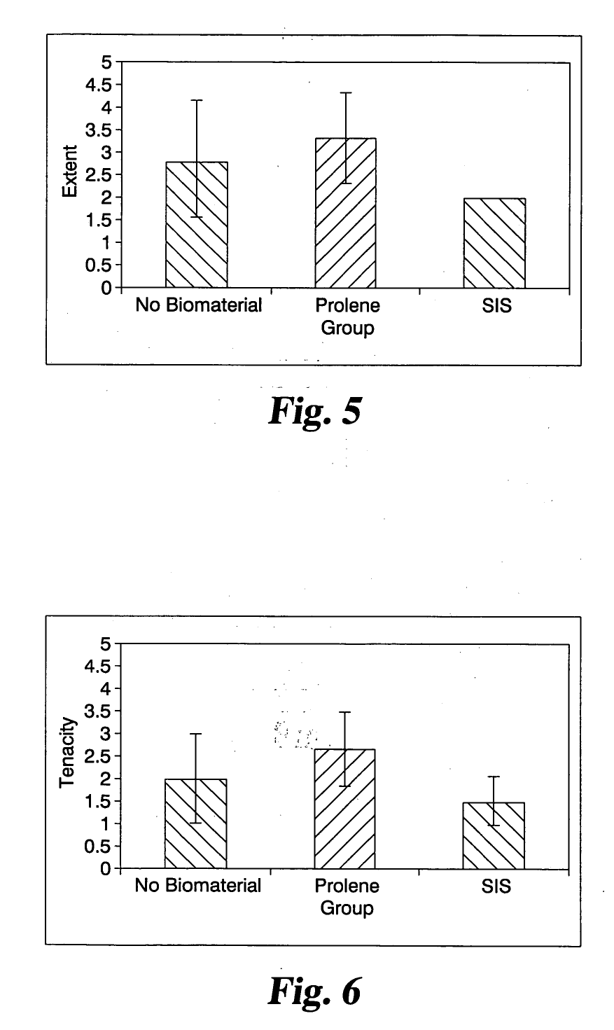

The results of the preliminary study are presented in Table 1. All the rats in the sham group survived the surgery. There were deaths due to the cecal abrasion injury in the 3 other groups: 3 in Group 2 (Control), 2 in Group 3, and 4 in Group 4. These deaths all occurred within 72 hours of the surgery, before adhesions would be expected to form or develop enough to be fatal. Mean extent and tenacity of adhesions within each treatment group is shown in Table 2 and FIGS. 5 and 6.

TABLE 1

| | | Body Mass | | |
|---|---|---|---|---|
| Treatment | Animal | (g) | Extent | Tenacity |
| Sham, No Abrasion | 1 | 233 | 0 | 0 |
| | 2 | 232 | 0 | 0 |
| | 3 | 215 | 0 | 0 |
| Abrasion, No | 1 | 281 | 4 | 3 |
| Biomaterial | | | | |
| | 2 | 265 | 3 | 3 |
| | 3 | 273 | 1 | 1 |
| | 4 | 274 | 4 | 2 |
| | 5 | 238 | 2 | 1 |
| Abrasion, then | 1 | 283 | 4 | 3 |
| Prolene | | | | |
| | 2 | 228 | 4 | 3 |
| | 3 | 268 | 4 | 3 |
| | 4 | 278 | 2 | 1 |
| | 5 | 275 | 4 | 3 |
| | 6 | 274 | 2 | 3 |
| Abrasion, then | 1 | 279 | 2 | 2 |
| SIS | | | | |
| | 2 | 282 | 2 | 1 |
| | 3 | 269 | 2 | 2 |
| | 4 | 273 | 2 | 1 |

The adhesions in the SIS group were all to mesentery except for animal #3, which had some adhesion to body wall. Adhesions in other groups were mostly to the body wall and mesentery.

Discussion

The study in this Example demonstrated differential adhesion extent and tenacity for the 4 treatment groups (p<0.01 for both; Kruskal-Wallis test). The data demonstrate that cecal abrasion in Sprague-Dawley rat causes significantly increased post-surgical adhesions when compared to sham operated controls (p<0.05 for extent and tenacity; Wilcoxan pairwise test).

Following cecal abrasion, implantation of Prolene mesh and SIS over the injured site caused significantly greater and more tenacious adhesions, compared to sham operated animals (p<0.05). However, both Prolene and SIS did not significantly decrease the number or strength of adhesions compared to injured animals that had no biomaterial implanted following injury. SIS significantly decreased the tenacity (p<0.05), but not the extent of adhesions compared to Prolene mesh.

As expected, cecal abrasions caused post-surgical adhesions to form. These adhesions were not prevented by the implantation of Prolene mesh or the 2-layer SIS construct. These results support the use of this animal model for the investigation of adhesion formation and prevention using tissue engineered scaffolds and anti-inflammatory drugs.

### EXAMPLE 2

### Nimesulide-Impregnated

### ECM Construct Inhibits Adhesion Formation

In this Example, the animal model described in Example 1 was used to test whether the addition of a NSAID compound to an SIS construct could be used to reduce post-surgical adhesion formation.

Materials and Methods

Forty-five strips of lyophilized 2-layer SIS measuring approximately 6 cm×2 cm were prepared from a single lot of standard strength SIS. The samples were randomly subdivided into 3 groups: “SIS” (mean SIS mass 79.6±7 mg), “High Dose Nimesulide” (74.9±10 mg), and “Low Dose Nimesulide” (75.2±11 mg). The “SIS” samples (without further treatment) were sterilized with ethylene oxide. The “High Dose” and “Low Dose” SIS constructs were soaked for 1 hour in 800 μM or 200 μM solutions of nimesulide (Sigma N1016, Lot 012K1278) in DMSO (100%, Sigma D5879, Batch 083K0136) respectively, at room temperature with moderate agitation. The samples were then removed from solution, frozen at −80° C. overnight, relyophilized, and sterilized with ethylene oxide.

Preliminary elution studies using mass spectroscopy to quantify the loading of nimesulide onto SIS using a DMSO vehicle were performed. These experiments on a very limited sample size demonstrated that a high dose sample contained 2.07±0.1 ng nimesulide/mg SIS, while a low dose sample contained 1.16±0.2 ng nimesulide/mg SIS. Extrapolation of these drug concentrations yields a dose range of 123-196 ng for the high dose samples and 68-105 ng for the low dose. These intraperitoneal administrations were well below the LD50 for rats (163 mg/kg).

Two sterile nimesulide/DMSO solutions were prepared in concentrations of 1.62 mM (0.5 mg/mL) and 6.49 mM (2.0 mg/mL), as was a 50 mL sterile aliquot of DMSO. Polypropylene mesh (Prolene, Ethicon, Lot TCB079) was purchased commercially.

Implantation Study.

Forty-nine 250-300 gram Sprague-Dawley rats were anesthetized with an IM dose of ketamine and xylazine and prepared for aseptic abdominal surgery. Using the techniques described in Example 1, the cecum was exteriorized and abraded with a nylon brush, producing peticheal bleeds. The adjacent wall of the peritoneal cavity was mildly abraded with the same nylon brush and the cecum was either placed back into the abdominal cavity, covered with biomaterial, which was anchored to the abdominal cavity by suturing it to mesenchymal fat, or injected with a solution and placed back into the abdominal cavity.

Following cecal abrasion, groups of rats received one of the following 8 treatments:
Treatment 1: (Control) No biomaterials were implanted following cecal injury. N=7 rats.
Treatment 2: (SIS) a lyophilized 2-layer piece of SIS was placed over the abraded surface and secured with non-absorbable nylon suture. Care was taken to avoid perforating the bowel N=6 rats.
Treatment 3: (PPM) a piece of polypropylene mesh (Bard) was placed over the abraded surface and secured with non-absorbable nylon suture. N=6 rats
Treatment 4: (SIS+high dose nimesulide) a lyophilized “High Dose” SIS material was placed over the abraded surface and secured with non-absorbable nylon suture. N=6 rats.
Treatment 5: (SIS+low dose nimesulide) a lyophilized “Low Dose” SIS material was placed over the abraded surface and secured with non-absorbable nylon suture. N=6 rats.
Treatment 6: (IP high dose nimesulide) 2.0 mg nimesulide in 1.0 mL DMSO (100%, Sigma D5879, Batch 083K0136) was delivered to the peritoneal cavity prior to closure. N=6 rats.
Treatment 7: (IP low dose nimesulide) 0.5 mg nimesulide in 1.0 mL DMSO was delivered to the peritoneal cavity prior to closure. N=6 rats.
Treatment 8: 11.0 mL DMSO was delivered to the peritoneal cavity prior to closure. N=6 rats.

Following placement of the cecum back into the abdominal cavity, the abdominal wall was closed with 4-0 silk and the skin closed with surgical staples. Twenty-one days after surgery, the rats were euthanized and the extent and tenacity of adhesions were evaluated in a blinded fashion using the scale and statistical analysis as described in Example 1.

Results

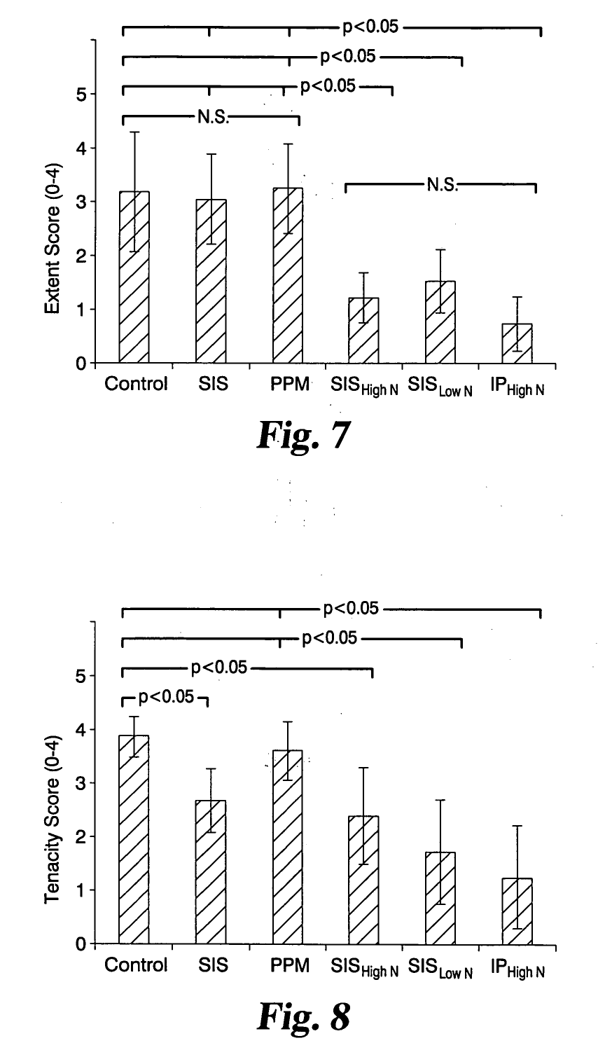

All the rats survived the surgery. Acute deaths (within 2 days) are summarized in Table 2. Mortality was 39%. There were deaths in all groups except control (no biomaterial implanted). Mean extent and tenacity of adhesions within each treatment group are shown in Table 3 and FIGS. 7 and 8. No deaths occurred during the remainder of the 21-day study.

TABLE 2

| Treatment Group | Acute Deaths (<2d) |
|---|---|
| 1 Control | 0 |
| 2 SIS | 2 |
| 3 Prolene Mesh | 1 |
| 4 SIS + high dose nimesulide | 1 |
| 5 SIS + low dose nimesulide | 2 |
| 6 IP high dose nimesulide | 2 |
| 7 IP low dose nimesulide | 5 |
| 8 DMSO | 6 |

TABLE 3

| Treatment | Extent | Tenacity |
|---|---|---|
| Control | 3 | 4 |
| | 3 | 4 |
| | 1 | 3 |
| | 3 | 4 |
| | 4 | 4 |
| | 4 | 4 |
| | 4 | 4 |
| SIS | 4 | 3 |
| | 3 | 3 |
| | 3 | 2 |
| Prolene Mesh | 3 | 3 |
| | 3 | 4 |
| | 2 | 4 |
| | 4 | 4 |
| | 4 | 3 |
| SIS + high dose | 2 | 3 |
| nimesulide | | |
| | 1 | 3 |
| | 1 | 1 |
| | 1 | 2 |
| | 1 | 3 |
| SIS + low dose | 1 | 1 |
| nimesulide | | |
| | 2 | 3 |
| | 2 | 2 |
| | 1 | 1 |
| IP high dose nimesulide | 1 | 2 |
| | 1 | 1 |
| | 0 | 0 |
| | 1 | 2 |
| IP low dose nimesulide | 2 | 2 |

A significant decrease in adhesion extent scores (FIG. 7) was found between groups with nimesulide versus groups without nimesulide (treatment groups 1-3 versus 4-6); a similar pattern of differences was shown for adhesion tenacity (FIG. 8). Deaths in groups 7 & 8 precluded inclusion of that data in the analysis.

Discussion

The work in this Example demonstrated differential adhesion extent between groups of treatments including no-biomaterial controls, 2-layer SIS, and Prolene mesh versus SIS augmented with nimesulide and IP injections of nimesulide. Tenacity of post-surgical adhesions followed a similar pattern, with the exception of a lack of statistically significant difference between SIS and SIS augmented with high dose nimesulide.

As demonstrated in Example 1, this surgical injury model was severe enough to cause a mortality rate of 39% among treated groups. It should be noted that all acute deaths occurred within 48 hours of surgery. This indicates that the likely cause of death was bowel ischemia, rather than post-surgical adhesions. In support of this, it was observed that there were no adhesions in one of the rats that had died following IP injection of DMSO vehicle.

Another aspect that may have led to high mortality was the use of DMSO as a delivery vehicle for nimesulide. Pure DMSO is extremely hygroscopic, and this may have led to critical peritoneal dehydration in the rats that received Treatments 7 & 8. This hypothesis is supported by the finding that post operative subcutaneous injection of normal saline following surgery prevented mortality in Group 6.

As expected, the cecal abrasion model of surgical injury caused post-surgical adhesions to form. These adhesions were not prevented by the implantation of Prolene mesh or 2-layer SIS. Addition of the anti-inflammatory drug nimesulide to SIS significantly attenuated adhesion extent and tenacity. This reduction in adhesions was not significantly different from the mean extent and tenacity of adhesions following treatment with IP injection of nimesulide. This Example supports that nimesulide can be used on an SIS construct to reduce the formation of post-surgical adhesions in the treatment of soft tissue injury with the SIS construct.

### EXAMPLE 3

### Testing with Additional Biomaterials

Materials

Twenty-seven strips of lyophilized 2-layer SIS measuring approximately 6 cm×2 cm were prepared from a single lot of standard strength SIS. This was the same material used in prior Examples. The samples were randomly subdivided into 2 groups: “SIS” (N=10, mean 80.4±15 mg) and “SIS+nimesulide” (N=17, mean 70.4±8 mg). The “SIS” samples (without further treatment) were sterilized with ethylene oxide. The “SIS+nimesulide” samples were soaked for 1 hour in an 800 μM solution of nimesulide (Sigma N1016, Lot 013K0925) in DMSO (Sigma D5879, Batch 083K0136, 30 mL & Sigma D1435, Batch 109H0036, 380 mL), at room temperature with moderate agitation. The samples were then removed from solution, frozen at −80° C. overnight, relyophilized, and sterilized with ethylene oxide. This treatment corresponds to the “SIS+high dose nimesulide” group in Example 2 above.

Preliminary elution studies using mass spectroscopy to quantify the loading of nimesulide onto SIS using a DMSO vehicle were performed. These experiments on a single sample of “SIS+high dose nimesulide” demonstrated that the sample contained 2.07±0.1 ng nimesulide/mg SIS. Extrapolation of this drug concentration yields a dose range of 113-174 ng for the animals treated with “SIS+nimesulide”. This dose range is well below the LD50 for intraperitoneal administrations in rats (163 mg/kg).

Polypropylene mesh (Prolene™, Ethicon, Lot TCB079) was obtained commercially and cut into forty-two 6 cm×2 cm strips. Twenty “Prolene mesh” and “Prolene mesh, IP nimesulide” samples (without further treatment) were sterilized with ethylene oxide. The remaining twenty-two strips were randomly assigned to the “Prolene+nimesulide” group (mean mass 111±8 mg). The “Prolene+nimesulide” samples were prepared by soaking for 1 hour in the same 800 μM solution of nimesulide in DMSO as the “SIS+nimesulide” samples (see above) at room temperature with moderate agitation. Most of the drug did not stick to the polypropylene mesh, as evidenced by the lack of yellow staining. The samples were then removed from solution, frozen at −80° C. overnight, relyophilized, and sterilized with ethylene oxide.

Ten 2 mg powder aliquots (mean mass 2.15±0.2 mg) of nimesulide were aseptically prepared and stored in 1.5 mL microcentrifuge tubes for later delivery to the peritoneal cavity following implantation of polypropylene mesh in the “Prolene mesh, IP nimesulide” treatment group.

Implantation Study

Forty-eight 250-300 gram Sprague-Dawley rats were anesthetized with an IM dose of ketamine and xylazine and prepared for aseptic abdominal surgery. Using established techniques as described in Example 1, the cecum was exteriorized and abraded with a nylon brush, producing peticheal bleeds. The adjacent wall of the peritoneal cavity was mildly abraded with the same nylon brush and the cecum was either placed back into the abdominal cavity or covered with material and then returned to the peritoneum.

Following cecal abrasion, groups of rats received one of the following treatments:
Treatment 1: (Control) No biomaterials implanted following cecal injury. N=8 rats.
Treatment 2: (SIS) a lyophilized 2-layer strip of SIS was placed over the abraded surface and secured with non-absorbable nylon suture. N=8 rats.
Treatment 3: (SIS+nimesulide) a lyophilized 2-layer SIS strip with nimesulide was placed over the abraded surface and secured with non-absorbable nylon suture. N=8 rats.
Treatment 4: (PPM) a strip of Prolene was placed over the abraded surface and secured with non-absorbable nylon suture. N=8 rats.
Treatment 5: (PPM+nimesulide) a strip of Prolene™ with nimesulide was placed over the abraded surface and secured with non-absorbable nylon suture. N=8 rats.
Treatment 6: a strip of Prolene was placed over the abraded surface and secured with non-absorbable nylon suture. 2 mg of nimesulide powder was delivered to the injury site. N=8 rats

SIS and Prolene™ mesh were anchored to the cecum by suturing them to mesenchymal fat. This enabled fixation of the materials without perforation of the bowel.

Following placement of the cecum back into the abdominal cavity, the abdominal wall was closed with 4-0 silk, and the skin closed with surgical staples. Twenty-eight days after surgery, the rats were euthanized and the extent and tenacity of adhesions were evaluated and statistics performed as in Example 1.

Results

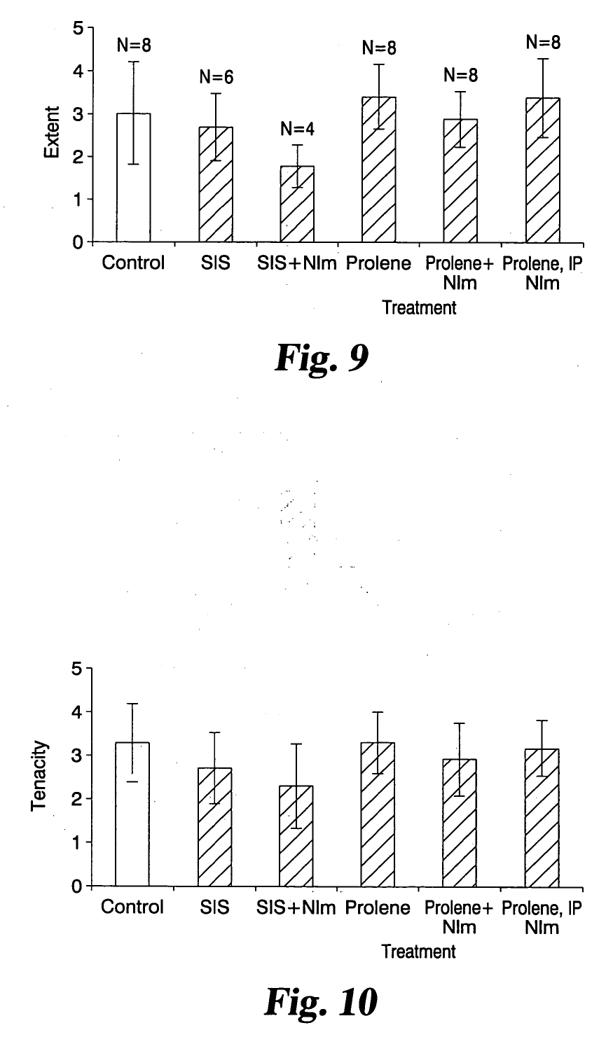

All the rats survived the surgery. Acute deaths (within 2 days) are summarized in Table 4. The mortality rate was lower than in previous studies using this model, which approached 40%. Mean extent and tenacity of adhesions within each treatment group are shown in Table 5 and FIGS. 9 and 10.

TABLE 4

| Treatment Group | Acute Deaths (<2d) |
|---|---|
| 1 Control | 0 |
| 2 SIS | 2 |
| 3 SIS + nimesulide | 4 |
| 4 Prolene mesh | 0 |
| 5 Prolene mesh + nimesulide | 0 |
| 6 Prolene mesh + IP nimesulide | 0 |

TABLE 5

| Treatment | Extent | Tenacity |
|---|---|---|
| 1 Control | 4 | 3 |
| | 4 | 2 |
| | 4 | 4 |
| | 2 | 4 |
| | 1 | 2 |
| | 2 | 3 |
| | 3 | 4 |
| | 4 | 4 |
| 2 SIS | 3 | 4 |
| | 4 | 3 |
| | 2 | 2 |
| | 3 | 2 |
| | 2 | 3 |
| | 2 | 2 |
| 3 SIS + nimesulide | 1 | 1 |
| | 2 | 3 |
| | 2 | 3 |
| | 2 | 2 |
| 4 Prolene mesh | 4 | 4 |
| | 3 | 3 |
| | 2 | 3 |
| | 4 | 3 |
| | 4 | 4 |
| | 3 | 3 |
| | 3 | 2 |
| | 4 | 4 |
| 5 Prolene mesh + nimesulide | 4 | 4 |
| | 3 | 3 |
| | 3 | 4 |
| | 2 | 2 |
| | 3 | 2 |
| | 2 | 3 |
| | 3 | 3 |
| | 3 | 2 |
| 6 Prolene mesh + IP | 2 | 3 |
| Nimesulide | | |
| | 4 | 4 |
| | 3 | 3 |
| | 4 | 3 |
| | 4 | 3 |
| | 2 | 3 |
| | 4 | 2 |
| | 4 | 4 |

There were no significant differences in adhesion extent or tenacity between groups in this first study. The mortality rate motivated the conduct of a replacement study. An issue presented by the first study was whether the lack of statistically significant differences between groups was a true effect or resulted from acute deaths in certain groups. The work reported in Example 2 demonstrated significant differences between Control, SIS, and Prolene versus nimesulide and SIS with nimesulide. The data from this initial study demonstrated a trend toward lower adhesion scores for SIS+nimesulide, but not to a significant level.

The replacement study was performed using identical methods to those of the initial study. The treatments in the replacement study are summarized in Table 6. Acute deaths occurred in all groups except control, with 1 animal dying in each of the SIS and SIS+nimesulide treatment groups. The post-surgical adhesion scores of the replacement animals are summarized in Table 7.

TABLE 6

| Treatment Group | Number of Animals |
|---|---|
| 1 Control | 5 |
| 2 SIS | 4 |
| 3 SIS + nimesulide | 8 |

TABLE 7

| Treatment | Extent | Tenacity |
|---|---|---|
| 1 Control | 4 | 4 |
| | 4 | 3 |
| | 3 | 3 |
| | 4 | 4 |
| | 3 | 4 |
| 2 SIS | 3 | 4 |
| | 3 | 3 |
| | 4 | 3 |
| 3 SIS + nimesulide | 3 | 4 |
| | 2 | 2 |
| | 2 | 2 |
| | 2 | 3 |
| | 3 | 3 |
| | 2 | 2 |
| | 1 | 3 |

Prior to combining the results of the replacement study those of the initial study, a Modified Levene's test (or Brown and Forsythe's Test) was performed to test for significant differences in the variance within each test group common between the 2 studies. These tests are less sensitive to the requirements for normality in a traditional F-test. Comparisons of the variances between studies revealed no significant differences.

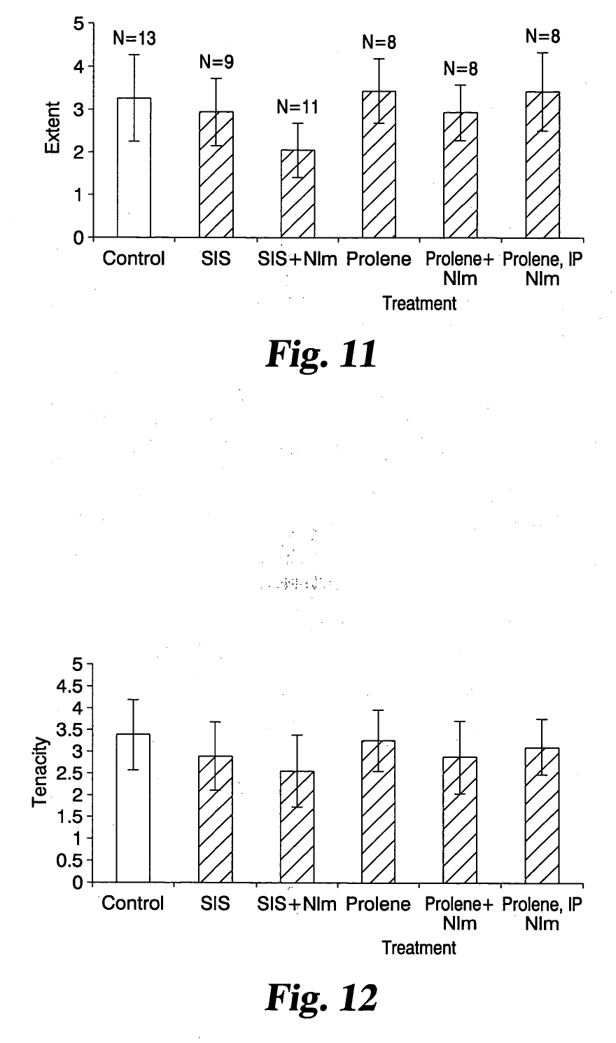

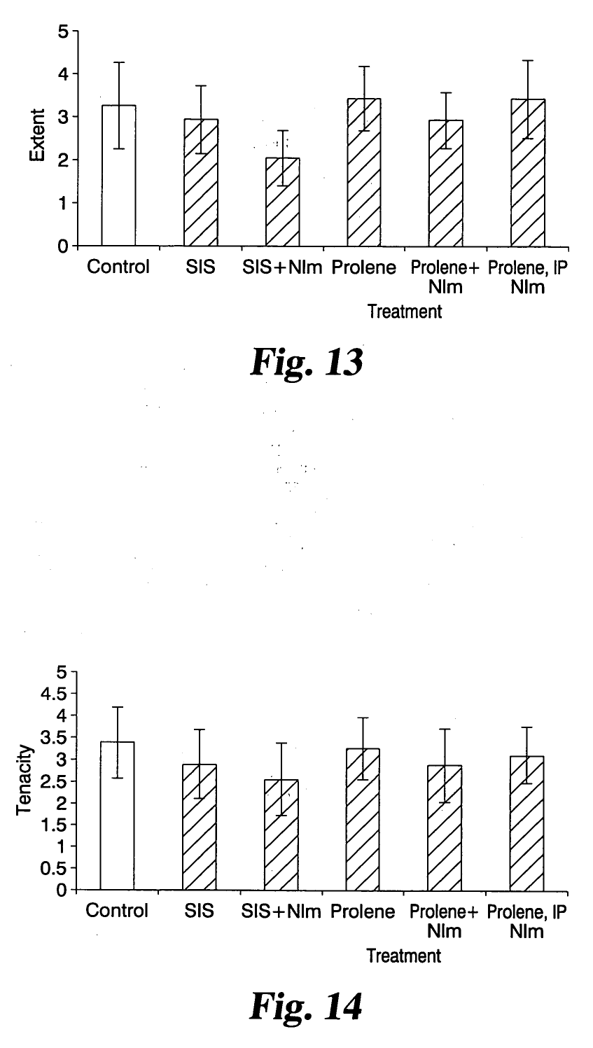

The replacement animal data were combined with the results of the initial study and are summarized in FIGS. 11 and 12. The pairwise Kruskal Wallis tests for adhesion extent and tenacity are presented in Tables 8 and 9 (in which “NM”=nimesulide) and are summarized visually in FIGS. 13 and 14.

TABLE 8

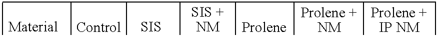

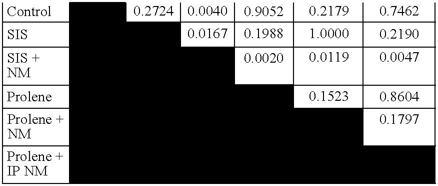

TABLE 9

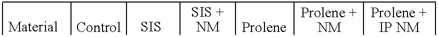

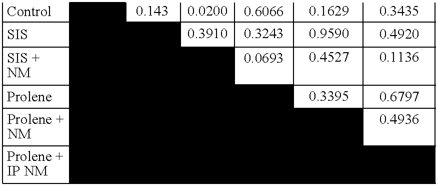

Discussion

As demonstrated in the previous Examples, the surgical injury model employed is severe enough to cause mortality rate of among treated groups. All acute deaths occurred within 48 hours of surgery. This indicates that the likely cause of death was bowel ischemia, rather than post-surgical adhesions. The acute deaths in the first study led to an additional study that replaced the losses. Before the studies were combined, statistical tests were performed to check for significant differences in variances within groups between the 2 studies. There were no significant differences and the studies were combined and analyzed by a non-parametric ANOVA.

In total, the current study demonstrated statistically significant differences in the adhesion extent score of group 3, SIS augmented with nimesulide and control, SIS, Prolene™ mesh, and Prolene™ mesh augmented with nimesulide. In tenacity scoring, SIS augmented with nimesulide scored significantly lower than controls, Prolene™, or Prolene™ with IP nimesulide.

### EXAMPLE 4

### Testing of Additional Compounds

In this example, additional compounds were tested for their ability to reduce tissue adhesions when applied to an SIS construct, using the animal model described in Example 1.

Materials and Methods

One hundred thirty-five 250-300 gram Sprague-Dawley rats were anesthetized with an IM dose of ketamine and xylazine and prepared for aseptic abdominal surgery. The cecum was exteriorized, gently abraded, and 9 groups of 15 rats each received the following treatments:
Control (no biomaterial implanted)
2-layer SIS biomaterial
2-layer SIS/nimesulide biomaterial
2-layer SIS/mitomycin C biomaterial
2-layer SIS/dexamethasone biomaterial
2-layer SIS/ibuprofen biomaterial
2-layer SIS/niflumic acid biomaterial
2-layer SIS/turmeric biomaterial
Seprafilm®, a sodium-hyaluronate-based bioresorbable membrane in clinical use as an adhesion barrier

Following placement of the cecum back into the abdominal cavity, the abdominal wall was closed with 4-0 silk, and the skin closed with surgical staples.

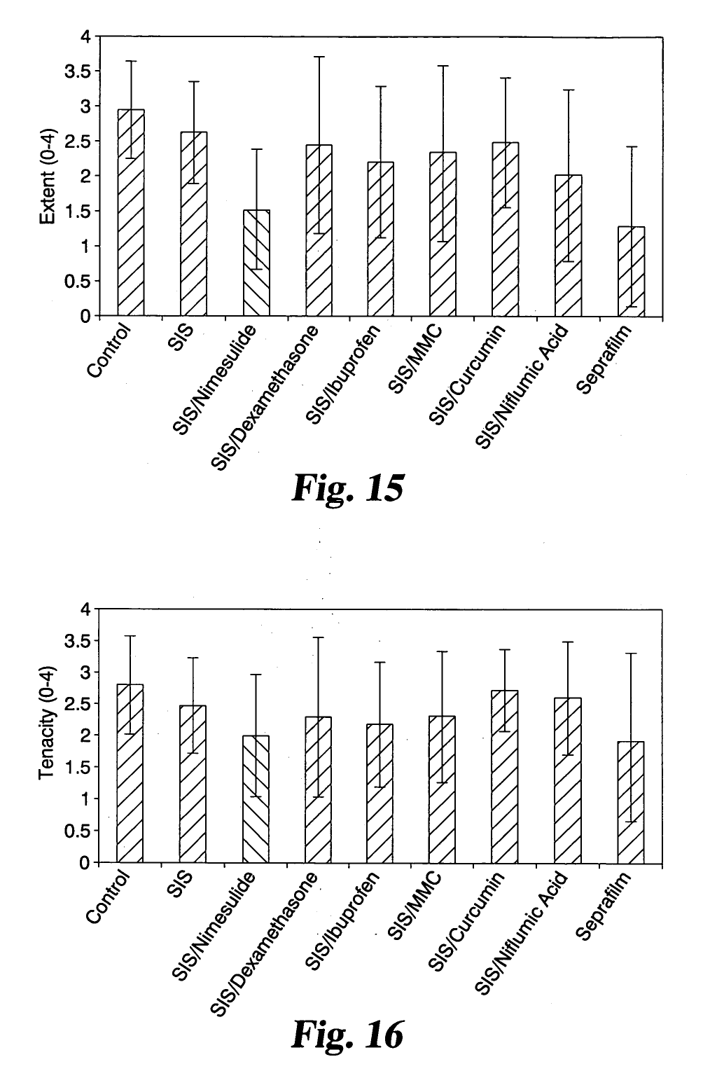

Twenty-one days after surgery, rats were euthanized and the extent and tenacity of adhesions were evaluated, and statistical analysis was performed, as in Example 1. Tissues were also sampled and fixed in formalin for histologic analysis. The results are shown in FIGS. 15 and 16, and demonstrate that other anti-inflammatory compounds can also be used to provide reductions in the extent and/or tenacity of tissue adhesions in the model.

While the foregoing specification teaches the principles of the present invention, with examples provided for the purpose of illustration, it will be appreciated by one skilled in the art from reading this disclosure that various changes in form and detail can be made without departing from the true scope of the invention.

All publications cited in the foregoing specification are hereby incorporated by reference in their entirety as if each had been individually incorporated by reference and fully set forth. Further, the attached document entitled “Addition of nimesulide to small intestinal submucosa biomaterial reduces post-surgical adhesions—A preliminary study” provides additional experimental details and is hereby made a part of this application, and all references therein cited are also hereby incorporated by reference in their entirety.

1. A medical implant material for providing tissue support at an implant site in a patient, comprising:
a remodelable extracellular matrix layer effective to promote tissue ingrowth into the layer; and
a non-steroidal anti-inflammatory drug (NSAID) carried by said remodelable extracellular matrix layer in an amount effective to inhibit the formation of tissue adhesions at the implant site.

2. The medical implant material of claim 1, wherein the NSAID is a selective COX-2 inhibitor.

3. The medical implant material of claim 1, wherein the NSAID is a selective COX-1 inhibitor.

4. The medical implant material of claim 1, wherein the NSAID is a non-selective COX inhibitor.

5. The medical implant material of claim 1, wherein the remodelable extracellular matrix layer comprises submucosa.

6. The medical implant material of claim 1, wherein the remodelable extracellular matrix layer retains at least one growth factor from a tissue from which it was derived.

7. The medical implant material of claim 1, which is a substantially planar sheet.

8. The medical implant of claim 7, wherein the NSAID is selectively incorporated on one side of the sheet.

9. The medical implant material of claim 7, wherein the NSAID is present on both sides of the sheet.

10. The medical implant material of claim 9, wherein the NSAID is distributed substantially homogeneously in the sheet.

11. The medical implant material of claim 1, wherein the sheet is a multilaminate construct.

12. The medical implant material of claim 11, wherein the construct is perforated.

13. The medical implant material of claim 2, wherein the NSAID is selected from the group consisting of nimesulide, diclofenac, etodolac, meloxicam, nabumetone, 6-MNA, celecoxib, and rolfecoxib.

14. The medical implant material of claim 13, wherein the NSAID is nimesulide.

15. The medical implant material of claim 3, wherein the NSAID is selected from the group consisting of flurbiprofen, ketoprofen, fenoprofen, piroxicam and sulindac.

16. The medical implant material of claim 4, wherein the NSAID is selected from the group consisting of aspirin, ibuprofen, indomethacin, ketorolac, naprosen, oxaprosin, tenoxicam and tolmetin.

17. A medical product, comprising:
a medical implant material according to claim 1; and
packaging enclosing said medical implant material in a sterile condition.

18. A medical mesh product for providing tissue support at an implant site in a patient, comprising:
a biocompatible layer for supporting tissue; and
an effective amount of a non-steroidal anti-inflammatory drug carried by said biocompatible layer to inhibit the formation of adhesions at said implant site.

19. The medical mesh product of claim 1, wherein the biocompatible layer is collagenous.

20. The medical mesh product of claim 1, wherein the biocompatible layer comprises a synthetic polymer.

21. The medical mesh product of claim 1, wherein the biocompatible layer comprises an extracellular matrix material.

22. The medical mesh product of claim 18, wherein the NSAID is a selective COX-2 inhibitor.

23. The medical mesh product of claim 18, wherein the NSAID is a selective COX-1 inhibitor.

24. The medical mesh product of claim 18, wherein the NSAID is a non-selective COX inhibitor.

25. The medical mesh product of claim 22, wherein the NSAID is selected from the group consisting of nimesulide, diclofenac, etodolac, meloxicam, nabumetone, 6-MNA, celecoxib, and rolfecoxib.

26. The medical mesh product of claim 23, wherein the NSAID is selected from the group consisting of flurbiprofen, ketoprofen, fenoprofen, piroxicam and sulindac.

27. The medical mesh product of claim 24, wherein the NSAID is selected from the group consisting of aspirin, ibuprofen, indomethacin, ketorolac, naprosen, oxaprosin, tenoxicam and tolmetin.

28. The medical mesh product of claim 25, wherein the NSAID is selectively incorporated on one side of the layer.

29. The medical mesh product of claim 26, wherein the NSAID is selectively incorporated on one side of the layer.

30. The medical mesh product of claim 27, wherein the NSAID is selectively incorporated on one side of the layer.

31. A medical material for delivery to an implant site in a patient to decrease tissue adhesions, comprising:
an implantable barrier layer; and
a non-steroidal anti-inflammatory drug (NSAID) carried by said barrier layer in an amount effective to inhibit the formation of tissue adhesions at the implant site.

32. The medical material of claim 31, wherein the barrier layer is biodegradable.

33. The medical material of claim 32, wherein the barrier layer comprises a biodegradable synthetic polymer.

34. The medical material of claim 31, wherein the barrier layer comprises an extracellular matrix material.

35. The medical material of claim 34, wherein the barrier layer comprises submucosa.

36. A method for providing tissue support, comprising:
implanting a medical mesh material at an implant site in a patient so as to provide tissue support, said medical mesh material comprising an effective amount of a non-steroidal anti-inflammatory drug carried by the material to inhibit the formation of tissue adhesions at the implant site.

37. A method for making an adhesion-inhibited medical tissue support mesh material, comprising:
providing a medical tissue support mesh material; and
incorporating on said medical implant material an effective amount of a non-steroidal anti-inflammatory agent to inhibit the formation of tissue adhesions on or adjacent to the material.

38. A method for decreasing tissue adhesions between an adhesiogenic tissue and at least one other structure, comprising:
implanting between the adhesiogenic tissue and the other structure a biocompatible barrier layer, the biocompatible barrier layer comprising an effective amount of a non-steroidal anti-inflammatory compound to decrease tissue adhesion formation between the adhesion-forming tissue and the other structure.

39. The method of claim 38, wherein the other structure is a tissue structure.

40. The method of claim 38, wherein the other structure is an implant material.

41. A method for providing tissue support and decreasing adhesions, comprising:
attaching a tissue-supporting medical mesh material to tissue to be supported, said tissue to be supported having adhesiogenic tissue adjacent thereto; and
interposing a biodegradable layer between said medical mesh material and said adhesiogenic tissue, said biodegradable layer incorporating an effective amount of a non-steroidal anti-inflammatory drug to reduce adhesions between said adhesiogenic tissue and said medical mesh material.

42. The method of claim 41, wherein said biodegradable layer is attached to said medical mesh material.

43. The method of claim 41, wherein said biodegradable layer is independent of said medical mesh material.

44. A method for prolonging the persistence of an implanted bioresorbable material in a patient, comprising incorporating an anti-inflammatory compound into the material.

45. The method of claim 44, wherein the material is a tissue support material.

46. The method of claim 44, wherein the material is a bioremodelable ECM material.

47. A method for reducing the rate of bioremodeling of a bioremodelable material, comprising incorporating an anti-inflammatory compound into the material.

48. The method of claim 47, wherein the material is a bioremodelable ECM material.

49. A tissue graft product, comprising:
a biocompatible implant material comprising a bioremodelable material; and
at least one anti-inflammatory compound selectively incorporated in and/or on a region of said bioremodelable material.

50. The product of claim 49, wherein said region is an external region.

52. The product of claim 49, wherein said region is an internal region.

53. The product of claim 50, wherein said external region is on a first face of said bioremodelable material, said bioremodelable material having a second face lacking said at least one anti-inflammatory compound.

54. The product of claim 49 wherein said biocompatible implant material comprises a multilaminate ECM construct.

55. A medical graft product, comprising:
a bioremodelable material incorporating an effect amount of an anti-inflammatory compound to reduce the rate of bioremodeling of the bioremodelable material when implanted in a mammal.

56. The medical graft product of claim 55, wherein said mammal is a human.

57. The medical graft product of claim 55, wherein the bioremodelable material is collagenous.

58. The medical graft product of claim 57, wherein the bioremodelable material is a collagenous, remodelable ECM material.

59. A method for tissue grafting, comprising:
implanting a bioremodelable material at a site in a patient; and
providing an anti-inflammatory compound at the site so as to reduce the rate of bioremodeling of the bioremodelable material.

60. The method of claim 59, wherein the anti-inflammatory compound is a non-steroidal anti-inflammatory drug (NSAID).

62. The method of claim 60, wherein the NSAID is a selective COX-2 inhibitor.

63. A method, material or product of any preceding claim, wherein the anti-inflammatory compound is water insoluble.

64. The method of claim 63, wherein the anti-inflammatory compound is an NSAID.
